(19) United States
(12) Patent Application Publication
(10) Pub. No.: US 2023/0148909 A1
(43) Pub. Date: May 18, 2023

(54) DAILY LIVING MONITORING AND MANAGEMENT SYSTEM

(71) Applicant: Baylor College of Medicine, Houston, TX (US)

(72) Inventor: Najafi Bijan, Houston, TX (US)

(21) Appl. No.: 17/995,540

(22) PCT Filed: Apr. 13, 2021
(86) PCT No.: PCT/US2021/027030

§ 371 Date: Oct. 5, 2022

Related U.S. Application Data
(60) Provisional application No. 63/009,702, filed on Apr. 14, 2020.

(51) Int. Cl. A61B 5/11; A61B 5/00

(52) U.S. Cl. CPC A61B 5/1113; A61B 5/1112; A61B 5/4088; A61B 5/6801; A61B 5/746; A61B 2562/0257; A61B 2562/0219

(57) ABSTRACT

A system for patient activity monitoring may include a wearable device and a personal terminal. The personal terminal may include a processor. The processor of the personal terminal may be configured to receive, from the wearable device, proximity detection information specifying proximity detected by the wearable device between the wearable device and a tag device and sensor data received by the wearable device from the tag device. The processor of the personal terminal may be further configured to determine, based on the proximity detection information and the sensor data, that an action was performed by a patient.

### CROSS-REFERENCE TO RELATED PATENT APPLICATIONS

This application claims benefit to the priority of U.S. Provisional Patent Application No. 63/009,702 filed on Apr. 14, 2020 and entitled “Patient Activity Monitoring And Management System,” which is hereby incorporated by reference herein.

### GOVERNMENT LICENSE RIGHTS

This invention was made with government support under Grant No. 1R42AG060853-01 awarded by the National Institute of Health. The government has certain rights in the invention.

### FIELD OF THE DISCLOSURE

The instant disclosure relates to medical monitoring and diagnostics. More specifically, certain portions of this disclosure relate to patient activity tracking and reminders.

### BACKGROUND

Dementia, Alzheimer's, and other chronic aging diseases are characterized by progressive cognitive and/or physical decline that may interfere with independent functioning. Dementia currently affects more than five million Americans and almost fifty million persons worldwide. The number is expected to increase to seventy-five million worldwide by 2030 and one-hundred and thirty-five million worldwide by 2050. In the United States alone the annual cost of dementia in 2017 was $259 billion and the annual cost is expected to exceed one trillion dollars over the next thirty years. There is no known cure for dementia, and caring for individuals with dementia requires a high level of collaboration among healthcare professions and caregivers.

An extensive network of healthcare and social service providers may provide care to individuals with chronic aging diseases, presenting challenges in coordination. However, coordination among healthcare professionals and caregivers has been shown to reduce symptoms, increase satisfaction with treatment, including reduction in depression and/or substance abuse. Furthermore, new treatment methods prioritize viewing the relationship between caregivers and/or medical professionals and patients as a collaborative relationship, where patients are encouraged to take charge of aspects of their treatment.

Patients experiencing cognitive decline may forget to perform necessary actions, such as activities of daily living, and may require additional assistance from caregivers to monitor completion of actions required for daily living. In some cases, decline may require that patients be placed in assisted living facilities to ensure that essential activities of daily living, such as bathing, eating, housekeeping, and other tasks, are completed. Furthermore, the medical, psychological, social, and functional aspects of chronic aging diseases may cause stress to patients, caregivers, and family of patients.

Shortcomings mentioned here are only representative and are included simply to highlight that a need exists for improved enhanced activity monitoring and reminder systems. Embodiments described herein address certain shortcomings, but not necessarily each and every shortcoming. Furthermore, embodiments described herein may present other benefits than, and be used in other applications than, those of the shortcomings described above.

### SUMMARY

A wearable, such as a necklace, pendant, or wrist device, may communicate with tags placed throughout a location, such as a home, to provide contextual activity information to a terminal device, such as a tablet, smart phone, personal computer, or central server. Contextual activity information may, for example, include information about activities of daily living (ADL) engaged in by a wearer of the wearable. When the wearable is brought, by a user, in proximity with a tag, proximity information may be transmitted to the terminal device notifying the terminal device that the wearable and the tag were brought within proximity of each other. The terminal device may determine, based on the proximity information, that a user has performed an action, such as engaging in one or more ADLs. For example, tags may be placed on various household items such as prescription medication containers, a shower, exercise equipment, doors, a stove, a refrigerator, a microwave, a water bottle, tables, chairs, beds, and other household items that users may approach and/or manipulate when engaging inactions, such as ADLs. A tag may be placed on a door to a shower or bathtub to detect when a user takes a shower or bath. A wearable worn by the user may sense proximity to and/or communicate with the tag and may communicate the proximity information for the tag and wearable to a terminal device. The terminal device may determine, based on the proximity information, that the user performed an action associated with proximity between the wearable and the tag. For example, the terminal device may determine that the user has taken a shower. The tags may sense other properties, such as movement of the tag, triggering of a button or switch on the tag, a voice of a user, or other occurrences indicating an activity, such as an ADL, has been performed or not performed by a user. For example, if a tag is placed on a faucet handle, the tag may sense motion indicating when the water is turned on or off, in addition to sensing proximity to the wearable. The sensed motion information may be transmitted to the wearable and/or the terminal device, and the device may, for example, determine when a user has forgotten to turn the water off. When determining that actions, such as taking a bath or shower or turning a faucet off, have not been performed, the terminal device may emit and/or may cause the wearable or one or more tags to emit, an audio, visual, or tactile alert to remind the user to complete the task. Thus, the system may provide enhanced monitoring of patients with aging diseases, as well as providing reminders to patients to perform required tasks. Such tracking and reminders can enhance treatment and caregiving outcomes and may provide family members with enhanced peace of mind. A system for monitoring patient actions, such as patient engagement in activities of daily living, may reduce the burden and stress of caregiving, increase patient adherence to scheduled actions, and improve independency and quality of life of patients.

Information regarding proximity of a first device, such as a wearable device, and a second device, such as a tag device, may be used to determine whether a user of the wearable device has performed one or more actions. An example method may begin with receipt, by a personal terminal, such as a tablet, laptop, or other personal terminal device, from a first device, such as a wearable device, a data log comprising proximity detection information specifying proximity detected by the first device between a second device, such as a tag device, and the first device. For example, when a user wearing a wearable device comes within a predetermined distance of a tag device the wearable device may detect and/or communicate with the tag to generate proximity information. For example, if a user wearing a wearable around their neck approaches or enters a shower or bath, the wearable may communicate with and/or detect a tag device on a door or faucet handle for the shower or bath and may generate proximity information in response. In some embodiments, proximity may be detected by the wearable device through a Bluetooth low energy (BLE) connection between the tag device and the wearable device or by detection, by the wearable device, of an RFID tag embedded in the tag device. In some embodiments, a camera or any other motion detectors may be used to detect motion and/or proximity between tags and users. The proximity information may be temporarily stored in a memory of the wearable device in a data log. The data log may then be transmitted from the first device to the personal terminal and may be received by a personal terminal device, such as a laptop, tablet, watch, smart phone, or other device. In some embodiments, the wearable device and/or tag device may transmit the proximity information to a remote cloud server, such as through a cellular or WiFi connection.

A determination may be made, based on the received proximity detection information, that an action, such as an activity of daily living, was performed by a patient. The determination that the action was performed by the patient may be used in health management of the patient. The terminal device may receive the data log including the proximity detection information and may determine, based on the data log, which devices, such as tag devices, the wearable was brought in proximity with. For example, if received proximity detection information indicates that the first device was brought in proximity with a second device attached to and/or associated with a refrigerator, the personal terminal may determine that the user has performed a mealtime action, such as eating breakfast, lunch, or dinner. If the received proximity detection information indicates that the first device was brought in proximity with a second device, such as a tag device, attached to and/or associated with a shower, the terminal device may conclude that the user has taken a shower.

In some embodiments, additional information may be received from the second device by the personal terminal. For example, user input information may also be received from the second device and stored in the data log of the first device. The user input information may include motion information related to motion of the second device, information related to triggering of a button of the second device, audio information, and other information. In some embodiments, the user input information may be transmitted directly from the second device to the terminal device, while in others, the user input information may be transferred from the second device to the first device, and then to the personal terminal. For example, the input information may be stored in the data log of the first device and transferred to the personal terminal with the data log. A determination that an action, such as an activity of daily living, was performed may be based on input information in addition to proximity information. For example, a second device, such as a tag device attached to and/or associated with a shower may include an accelerometer and/or a gyroscope to detect movement. When a door of the shower is opened, the second device may generate input information based on movement of the shower door. The personal terminal may determine that a user opened the shower door based on proximity information and input information. In another example, a combination of interactions with several tagged objects may be used to detect that activity of daily living was engaged in by the user. For example, when a user interacts with several tagged objects inside of a kitchen, such as a refrigerator, one or more kitchen cabinet doors, an oven, etc., the combination of interactions may be used to determine that the user is cooking or has cooked. In another example, when the user interacts with tagged objects inside of a house, such as a vacuum cleaner, a washer, or a dryer, the combination of interactions may be used to determine that the user is performing a housekeeping task. Combinations of interactions that indicate performance by a user of particular tasks, such as activities of daily living, may be predetermined or learned by a machine learning algorithm to define a set of criteria or rules that are executed either locally to the user, such as on a personal terminal, or on a remote server analyzing the data of collected interactions to determine characteristics of the user, such as cognitive capacity. Use of both proximity information and input information can help to avoid false reporting of actions and can enhance health management of the patient. For example, if a person other than the patient opens the shower door to take a shower, the personal terminal may note the absence of reported proximity information for the wearable and the tag device and may determine that the user has not taken a shower. Likewise, if the patient approaches the shower, generating proximity information, but does not open the shower door, the personal terminal may note the absence of input information and may determine that the patient has not taken a shower.

In some embodiments, a lack of input information or proximity information can be used to remind the user to complete a scheduled task. For example, when the user interacts with a tagged door of a microwave and an activity of placing an object inside of the microwave is detected, but an activity of removing the object from the microwave is not detected within a predefined period of time, a reminder may be sent to the user take the object out of the microwave. For example, a reminder to remove the object from the microwave may be sent to a smart phone of the user, in the form of a message, or to the wearable of the user, in the form of haptic feedback, an audio alert, a visual alert, or another alert. Similarly, the lack of input information may help to detect and alert a dangerous scenario. For example, when the user interacts with a tagged stove or oven and an activity of turning the stove on was detected, the user may be sent a reminder alert to ensure the stove is turned off or to check whether the stove is operating correctly. In some embodiments a number of interactions between the user and a tag device may be used to detect a disease. For example, a number of times the user interacts with a toilet tag within a pre-defined time interval may be used to determine that the user is subject to a urinary tract infection.

Information detailing performance of activities of daily living, life space size, adherence to scheduled tasks, and/or other information, may be used to determine or track cognitive capacity of the user. For example, when a life space size of the user, such as an area in which a user operates during their day to day life, is shrinking from moving between several rooms inside of home to a life space size approximately a distance between two particular locations, such as bedroom and bathroom, an alert may be generated to indicate a decline in the user cognitive capacity. Similarly, information detailing performance of actions, such as activities of daily living, may be used to track changes in cognitive capacity. For example, when a number of activities of daily living such as cooking, housekeeping, taking medication, care of pets, etc., performed per day shrinks for several consecutive days an alert may be generated to indicate decline in cognitive capacity. In some embodiments, a frequency engagement in activities of daily living may be used to detect a clinical condition. For example, when a frequency of using a bathroom increases beyond of a predefined threshold, an alert may be generated for a potential sign of unitary tract infection condition. In some embodiments, the geographic location of the tag devices inside and outside of home is used to determine the user life space size. For example, the interaction of the user with different tag objects inside of the home is used to determine which rooms or locations inside of home the user has navigated each day. Interaction with a car tag and an exit door tag may be used to determine how many times the user left home. Use of a smart phone tag may be used to determine distances from home the user has travelled each day or over a series of days. The longest distance travelled per day and diversity of locations inside and outside home traveled by the user in a day, or over another period of time, may be used to determine life space size. The determined life space size may be used to determine functional and cognitive performance of the user. For example, if the life space size is limited to a bedroom and bathroom, such limitation may indicate poor cognitive function. However, if the life space size includes a wide range of geographic locations inside of home such as bathroom, living room, laundry room, and kitchen, frequent departure from the home, and/or travelling far distances from home, cognitive function may be assessed to be healthy.

A first device, such as a wearable device, may transmit information in addition to proximity information to the personal terminal. For example, the first device may sense motion, voice activity, location, heart rate, temperature, audio, CO₂, and other biometric and user data and may transmit such data to the personal terminal. Likewise, the first device may transmit user input information received from the second device in a data log. Such data may be further analyzed by the personal terminal to determine when actions are performed by a user. In some embodiments, additional sensors may be installed inside of the user's home to detect indoor air quality. For example, a sensor placed inside of the kitchen may detect presence of CO₂ when the user interacts with a tagged stove. Detection by such sensors may be used to generate an alert to remind the user or his/her caregiver to check the stove for correct operation or to turn off the stove.

A patient and/or his/her caregiver(s) may be alerted of actions to be performed. For example, a personal terminal may determine that a scheduled action, such as taking a morning medication, is scheduled to be performed. The personal terminal may alert the patient of the action. In some embodiments, a caregiver may configure the personal terminal to generate reminders for scheduled actions at predetermined times. Alerts generated by the personal terminal may include visual alerts, displayed on a display of the terminal device, audio alerts emitted by a speaker of the personal terminal, and/or a vibration alert via a watch and/or other wearable, mobile, or other electronic devices. Alternatively or additionally, the personal terminal may cause the wearable device, a tag device, or a peripheral device such as a smart speaker, to emit, display, or otherwise generate an alert. The personal terminal may then wait for receipt of a data log including information, such as proximity information, from the first device and/or one or more second devices indicating that the scheduled action was performed. If the action is not performed by the patient within a predetermined time frame, the personal terminal may transmit an alert to a control terminal and/or generate additional alerts. The control terminal may, for example, be a server on the cloud or other remote terminal. The alert may then be pushed to a web-based application, a mobile application, or other system, such as a text messaging system, to alert a caregiver, family member, or medical professional that the scheduled task was not performed.

The steps described herein may be included in code of a computer program product for execution by a computing device to carry out certain steps of the disclosure. For example, a processing station may execute a computer program to perform steps of receiving and determining, as disclosed herein. Furthermore, an apparatus may include a memory and a processor for performing the steps described herein. A system for action detection may include a first device, such as a wearable device, a second device, such as a tag device, and a personal terminal having a processor for performing the steps described herein. The first device may be configured to detect proximity between the first device and the second device. The first device may comprise a first sensor for sensing proximity between the first device and the second device. The second device may comprise a second sensor for sensing motion of the second device. The second sensor of the second device, may, for example, be a motion sensor, a button, or a switch. The first sensor of the first device may, for example, be a BLE communication module or an RFID tag reader.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### BRIEF DESCRIPTION OF THE DRAWINGS

For a more complete understanding of the disclosed system and methods, reference is now made to the following descriptions taken in conjunction with the accompanying drawings.

FIG. 1A is an illustration of a wearable sensing device worn by a user according to some embodiments of the disclosure.

FIG. 1B is an illustration of a wearable sensing device connected to a personal computer according to some embodiments of the disclosure.

FIG. 2A is a bar graph of monitored steps during a twenty-four hour period for cognitive intact individuals and cognitive impaired individuals according to some embodiments of the disclosure.

FIG. 2B is a bar graph of monitored walking time during a twenty-four hour period for cognitive intact individuals and cognitive impaired individuals according to some embodiments of the disclosure.

FIG. 2C is a bar graph of a number of transitions from standing to sitting during a twenty-four hour period for cognitive intact individuals and cognitive impaired individuals according to some embodiments of the disclosure.

FIG. 3 is a diagram a number of minutes of activity in a twenty-four hour period for individuals with varying mental and physical states according to some embodiments of the disclosure.

FIG. 4 is a diagram of a system for action reminders, monitoring, and reporting, according to some embodiments of the disclosure.

FIG. 5 is a block diagram of a tag device, wearable device, personal terminal, and cloud in communication with each other, according to some embodiments of the disclosure.

FIG. 6 is a flow chart of an example method for monitoring user actions according to some embodiments of the disclosure.

FIG. 7 is a flow chart of an example method for notification of missed actions according to some embodiments of the disclosure.

### DETAILED DESCRIPTION

Individuals with chronic aging diseases, such as dementia and Alzheimer's, may experience cognitive and physical decline. A network of caregivers and medical professionals may be engaged in care of such individuals. Care of individuals with chronic aging diseases may involve substantial co-operation between patients, caregivers, and medical professionals to manage treatment and activities of daily living. An integrated system to support self-care, assist caregivers with care coordination, and facilitate patient data collection, aggregation, and sharing amongst caregivers and medical professionals may improve patient quality of life and treatment outcomes, may reduce the workload of caregivers, and may provide greater peace of mind to family members. Such a system may allow caregivers and/or medical professionals to schedule caregiving tasks, monitor physical activity, such as completion of scheduled actions, and facilitate timely completion of scheduled actions, such as taking medications, toileting, hydration, meals, and attendance at scheduled appointments.

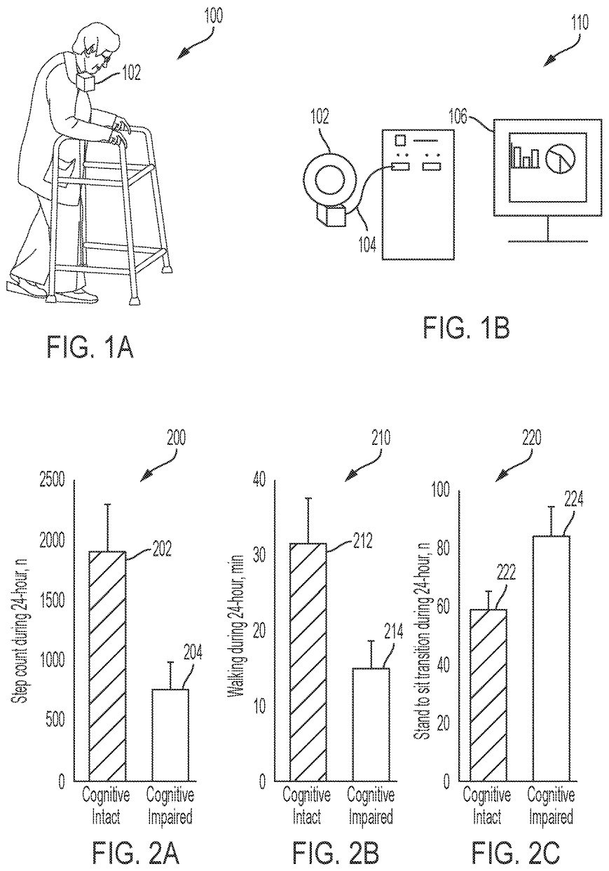

Activity of individuals with chronic aging diseases, such as engagement in activities of daily living, may be monitored using wearable devices. For example, a patient 100, as shown in FIG. 1A, may wear a wearable device 102. The wearable device 102 may be worn around the neck of a patient, around a wrist of a patient, around an ankle of a patient, or on another body part of the patient. The wearable device 102 may include internal sensors such as an accelerometer, a tri-axial gyroscope, a heart rate sensor, a microphone, and other sensors for monitoring activity of the patient. Some wearable devices may include internal memory for recording activity of the patient for analysis by caregivers and/or medical professionals. For example, the wearable device 102 may collect information while worn by a patient and may be connected to a personal computer 106 as shown in desktop setup 110 of FIG. 1B to transfer collected information to the personal computer 106. For example, the wearable device 102 may be connected to a personal computer 106 via a universal serial bus (USB) connection 104, or other wired connection, to transfer data from the memory of the wearable device 102 to a personal computer 106 for analysis by a caregiver or medical professional. A requirement for a physical connection to transfer data, however, may be inconvenient, requiring the wearable to be removed from a patient for data transfer. Further, requiring a physical connection for data transfer may make real time updates as to patient activity impracticable, thus reducing a level of patient care. Thus, wireless connectivity between a wearable and a terminal device, such as a tablet, personal computer, or remote server, may be desirable. Furthermore, while a wearable device can sense motion data, a wearable device alone may provide less contextual information regarding user actions than may be provided by other embodiments described herein.

Patient activity data sensed by a wearable device, such as motion data, may be used to monitor patient activity metrics, such as a number of steps taken by a patient in a day, a number of minutes spent walking in a day, a number of transitions between standing and sitting during a day, and other activity metrics. Patient activity data may, for example, indicate patient engagement in activities of daily living, which may provide insight into patient physical and cognitive health. In particular, patient activity metrics can be used to monitor physical and/or cognitive health of patients. For example, the bar graph 200 of FIG. 2A contrasts a number of steps taken in a 24-hour period by an individual with cognitive impairment and an individual without cognitive impairment. A number 202 of steps taken by an individual without cognitive impairment may be approximately 1900, substantially greater than a number 204 of steps taken by an individual with cognitive impairment, around 700. The bar graph 210 of FIG. 2B contrasts a number of minutes spent walking during a 24-hour period by an individual with cognitive impairment and an individual without cognitive impairment. A number 212 of minutes spent walking during a 24-hour period by an individual without cognitive impairment may be approximately 30, substantially greater than a number 214 of minutes spent walking by an individual with cognitive impairment, approximately 15. The bar graph 220 of FIG. 2C contrasts a number of stand to sit transitions during a 24-hour period by an individual with cognitive impairment and an individual with cognitive impairment. A number 222 of stand to sit transitions during a 24-hour period by an individual without cognitive impairment may be approximately 60, substantially lower than a number 224 of stand to sit transitions by an individual with cognitive impairment, approximately 85.

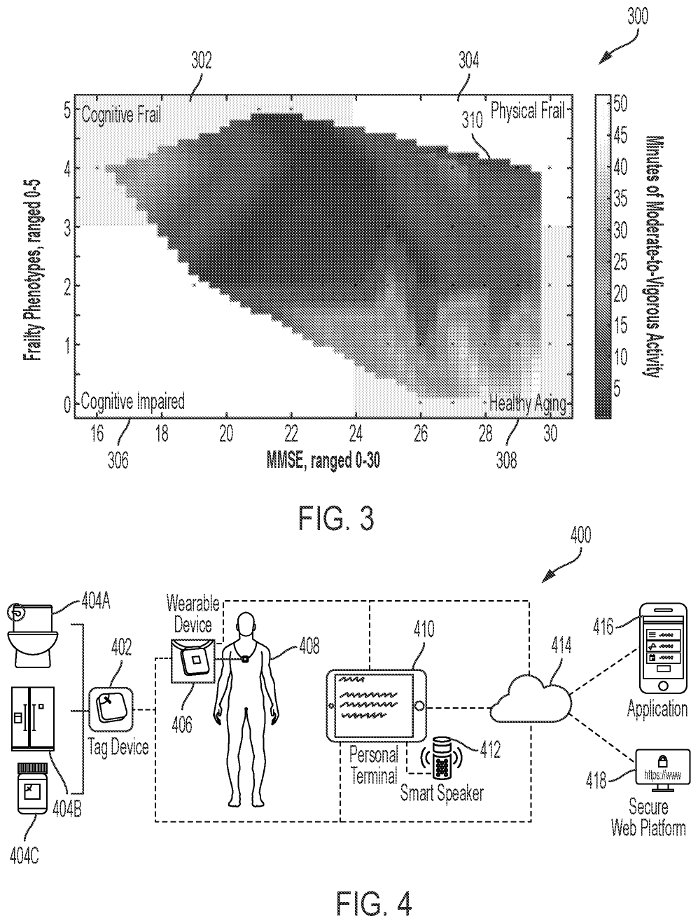

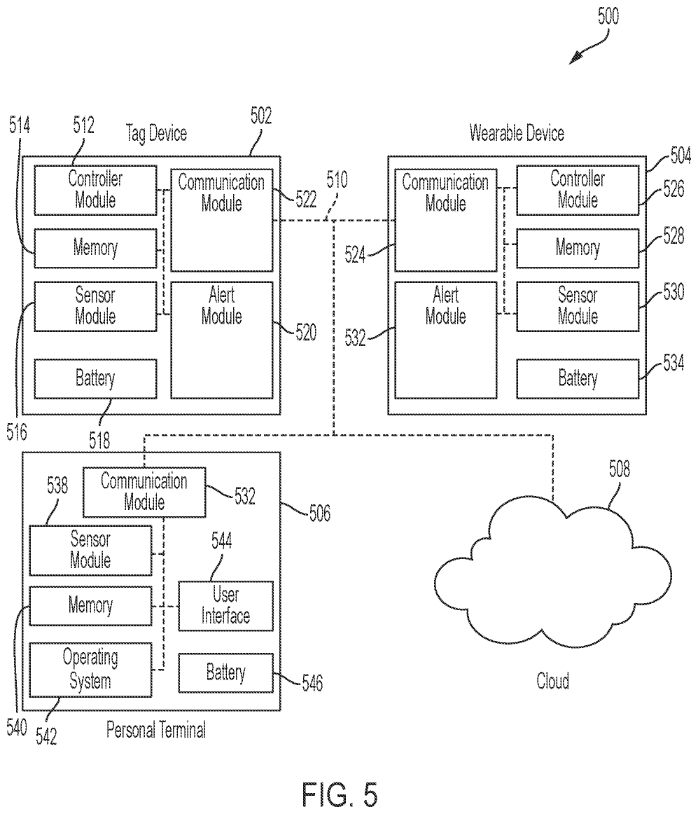

A number of minutes of moderate-to-vigorous physical activity in a twenty-four hour period may vary based on physical and cognitive health of a patient. For example, the graph 300 of FIG. 3 shows a plot of moderate-to-vigorous physical activity over a twenty-four hour period for a number of individuals with varying levels of physical and mental frailty. The y-axis of the graph 300 represents progressive physical frailty phenotypes, ranging from zero to five with progressing physical frailty. The x-axis represents mini-mental state examination (MMSE) scores ranging from sixteen to thirty, with higher scores representing more healthy cognitive states. Lower right quadrant 308 includes data points for individuals with moderate to high physical health and little to no cognitive impairment. As shown by the graph 300, individuals with high physical health and little to no cognitive impairment engage in high levels of moderate to vigorous activity, approximately thirty to sixty minutes per day. The lower left quadrant 306 includes individuals with moderate to high levels of cognitive impairment, who are physically non-frail. As shown by the graph 300, individuals with moderate to high levels of cognitive impairment engage in lower levels of physical activity, sometimes less than ten minutes per day. The upper left quadrant 302 includes data points from individuals with moderate to high levels of cognitive impairment and high levels of physical frailty. As shown by the graph 300, such individuals engage in relatively low levels of physical activity as well. The upper right quadrant 304 includes data points from individuals with little to no cognitive impairment and high levels of physical frailty. Such individuals also engage in relatively low levels of physical activity. Thus, individuals that have low levels of physical frailty and cognitive impairment tend to engage in more moderate-to-vigorous physical activity than individuals with higher levels of physical frailty and cognitive impairment.

Patients being treated for chronic aging diseases, such as dementia or Alzheimer's, may be served by a network of caregivers and medical professionals. Additional information about patient activity may enhance the quality of care provided by caregivers and medical professionals, while also providing ease of mind to family members of such patients. In particular, insight into patient completion of or engagement in activities of daily living may allow caregivers and medical professionals to enhance a quality of care given to a patient and an overall quality of life of the patient. Furthermore, such a system may enable greater patient management of activities of daily living, reducing the burden on caregivers and medical professionals. For example, a system for monitoring activity of patients may also be used to remind patients to complete activities of daily living, such as taking medications, eating meals, bathing, and use of the facilities and to notify caregivers when scheduled activities are not completed. An example system 400 for user/patient monitoring and notification is shown in FIG. 4. The system 400 may include a wearable device 408, one or more tag devices 402, a personal terminal 410, one or more cloud servers 414, a mobile application 416, and a secure web platform 418.

A user 408 may wear a wearable device 406 to track user activity, such as user engagement in and/or completion of activities of daily living. The wearable device 406 may, for example, monitor motion of the user, a heart rate of the user, a voice of the user, and other user activity and/or characteristics. The wearable device 406 may be worn around a neck of the user 408, on a wrist of the user 408, or on another body part of the user 408. The wearable device 406 may include wireless connectivity capability. For example, the wearable device 406 may communicate wirelessly, such as using a Bluetooth, WiFi, or cellular connection, with a personal terminal 410, such as a tablet, smart phone, or personal computer to transfer data from the wearable device 406 to the personal terminal 410. The wearable device 406 may include a Bluetooth low energy (BLE) module, RFID tag reader, or other wireless connectivity module to detect and/or connect with tag devices 402 when brought within proximity of the tag devices 402. In some embodiments, the wearable device 406 may communicate with a remote server in the cloud 414 via a WiFi or cellular connection. The wearable device 406 may also include alert capabilities, for alerting the user to user actions that should be performed. Such alerts may include visual alerts, audio alerts, tactile alerts, and other alerts. For example, the wearable device 406 may alert the user 408 when it is time to take a shower, may alert the user 408 to turn off the water when the shower is over, if the user 408 forgets, may alert a user 408 when it is time to take a prescription medication, and may provide other alerts.

One or more tag devices 402 may be placed around a home of the user 408 and may be used in coordination with the wearable device 406 to determine when a user has performed an action, such as an activity of daily living. For example, a tag device 402 and the wearable device 406 may include proximity sensors to determine when the wearable device 406 is moved within a predetermined distance of the tag device 402. In some embodiments, tag devices 402 may be placed outside a home of a user as well, such as in a medical office or in a vehicle of a user. In some embodiments, the tag device 402 may include an RFID tag that is detected by the wearable device 406 when the wearable device 406 and tag device 402 are brought within proximity of each other. Alternatively or additionally, the wearable device 406 and tag device 402 may include BLE modules for connecting to each other and generating proximity data when the devices are brought within proximity of each other. Thus, the wearable device 408 may detect when a tag device 402 enters proximity with the wearable device 408 and may determine that an action was performed based on the detection of the tag device 402.

For example, a tag device 402 may be attached to a toilet 404A to determine when a user 408 of the wearable device 406 has used the toilet 404A. In some embodiments, the tag device 402 may include additional sensing modules, such as a button, a switch, an accelerometer, a gyroscope, or a microphone. For example, the tag device 402 may be attached to a handle of the toilet 404A and may sense motion data when the handle of the toilet 404A is triggered. Thus, if a user 408 is detected as using the toilet 404A, due to proximity detected between the wearable device 406 and the tag device 402, but is not detected as flushing the toilet 404A, due to lack of motion detected by the tag device 402, the wearable device 406, or the personal terminal 410, may generate an alert, such as an audio or tactile alert, to remind the user to flush the toilet 404A.

As another example, a tag device 402 may be attached to a refrigerator 404B. The wearable device 406 and tag device 402 attached to the refrigerator 404B may, for example, allow for detection of a user opening the refrigerator and eating a meal. The tag device 402 may detect proximity of the wearable device 406 and may also detect motion when the user 408 opens a door of the refrigerator 404B. In some embodiments, the wearable device 406 may provide a reminder to the user 406 to eat a meal when proximity between the wearable device 406 and a tag device 402 attached to the refrigerator 404B has not been detected. As another example, a tag device 402 may be attached to a prescription medication 404C to detect when the user 408 has taken the prescription medication 404C. The wearable device 406 may also remind the user 408 to take the prescription medication 404C when the user 408 has not done so within a predetermined time frame. Tag devices may be coupled to other household devices, such as doors, to detect when a user has opened a door, microwaves, ovens, clothing items, such as compression socks, vehicles, beds, chairs, tables, and other items to allow the wearable device 406 to detect proximity of the user 408 to such items. In some embodiments, the tag devices 402 may communicate directly with a personal terminal 410 via a Bluetooth, WiFi, cellular, or other wireless connection. In some embodiments, the tag devices 402 may only communicate with the wearable device 406. For example, the tag devices 402 may communicate proximity information, and, in some embodiments, user input information such as motion information or triggering of a button or switch on the tag device, when the wearable device 406 is in close proximity with the tag device 402, such as via a BLE connection.

The personal terminal 410 may, for example, be a laptop, tablet, smart phone, smart watch, or other terminal device. In some embodiments, the personal terminal 410 may be a device, such as a tablet, that is specially configured to manage the user's health. In other embodiments, the personal terminal 410 may be a general use personal device, such as a tablet, with an application for managing the user's health. The personal terminal 410 may receive activity information from the wearable device 408 and/or one or more tag devices 402, such as proximity information, motion information, user biometric information, such as heart rate, temperature, and voice information, and user input information, such as information regarding user motion of a tag device or pressing a button on the tag device. The personal terminal 410 may analyze the information received from the wearable device 406 and/or one or more tag devices 402 to determine when the user 408 has performed one or more actions. For example, the personal terminal 410 may analyze proximity information received from the wearable device 406 and/or a tag device 402 attached to a prescription medication to determine that the user 408 took the prescription medication. In some embodiments, the personal terminal 410 may receive user input information, such as motion information or information regarding triggering of a button or switch on the tag device 402, directly from the tag device 402, while in other embodiments the personal terminal 410 may receive the user input information via the wearable device 406, after the user input information is transferred from one or more tag devices 402 to the wearable device 406.

In some embodiments, the voice of the user during performed tasks is measured to detect the cognitive performance of the user. For example, a duration of simultaneous walking and talking may be sensed to determine cognitive function of the user. In another example, a number of times per day that the user interacts with a voice-enabled device, such as a voice-enabled security camera, voice-enabled lighting system, or voice-enabled smart home device, may be sensed and recorded. Changes in a user's voice interactions with smart devices over time may be used to evaluate cognitive performance. Voice interaction with other objects inside of the home may also serve as a method for reinforcement of adherence to predefined tasks. For example, a voice response to an audio reminder or a text reminder may be used to determine compliance in performing a task. An audio reminder may be programmed via one or more voice-enabled devices.

The personal terminal 410 may also provide reminders to a user of scheduled activities. For example, at a predetermined time, if the personal terminal 410 has not detected that the user has eaten breakfast, the personal terminal 410 may trigger a reminder to the user to eat breakfast. Reminders triggered by the personal terminal 410 may include visual reminders displayed by the personal terminal 410 or audio reminders emitted by the personal terminal 410. Alternatively or additionally, the personal terminal 410 may cause reminders to be displayed by or emitted by other devices, such as a smart speaker 412. Reminders unrelated to activity detection may also be displayed by the personal terminal 410, such as reminders for calls or appointments with caregivers or medical professionals. In some embodiments, the personal terminal 410 may display, emit, or otherwise generate reminders based on detected actions. For example, if the personal terminal 410 determines that a user has taken and left a shower, but has not turned the water off, based on a lack of motion data received from a tag device 402 attached to a faucet handle of the shower, the personal terminal may emit an audio alert, or may cause the wearable device emit an audio alert to remind the user to turn the water off. In some embodiments, the personal terminal 410 may cause the wearable device 406 to generate a tactile alert, such as vibration. The personal terminal 410 may communicate with a remote cloud server 414. For example, data received from the wearable device 406 and/or tag devices 402 and determinations of actions made by the personal terminal 410 may be uploaded to a remote server 414. The personal terminal 410 may also include voice recognition functionality to receive voice input from a user. For example, user responses to reminders may be recorded by the personal terminal and uploaded to the a remote server 414 on the cloud.

The remote server 414 may aggregate activity information related to a user 408, making such information accessible to caregivers and/or medical professionals. For example, activity information may be made accessible for caregivers and/or medical professionals through a secure application 416, such as a smart phone application, a desktop application, a tablet application, or other application, or through a secure web platform 418. In one example scenario, an alert for an application 416 or secure web platform 418 may be generated by the server 414 or the personal terminal 410 when a determination is made that a user has not taken a shower or has not eaten a meal by a predetermined time. In some embodiments, an alert may be generated for the application 416 or the secure web platform 418 when the personal terminal 410 determines that a user has performed a first action, but has not performed a second action that is required to be performed following the first action. For example, if a determination is made that a user has taken a shower, but the personal terminal 410 has not detected the water for the shower being turned off, an alert may be generated on an application 416 or secure web platform 418 accessible by a caregiver to inform the caregiver that the water has not been turned off. In some embodiments, a caregiver may be able to communicate with the user 408 via the application 416 or secure web platform 418 by voice or text. For example, the caregiver may speak with the user via an audio connection between the application 416 and the personal terminal 410 and/or the smart speaker 412 and the wearable device 406. A caregiver or medical professional may also generate alerts and/or schedule actions for the user 408 using the application 416 or the secure web platform 418. In some embodiments, the application 416 and/or secure web platform 418 may allow caregivers and/or medical professionals to coordinate care, view physical activity and action information, and schedule reminders for users to perform actions.

The secure application 416 may, for example, include a user interface operating on a VueJS framework, with a NativeScript framework used to provide a native mobile application for Android and/or iOS using JavaScript and Cascading Style Sheets (CSS). In some embodiments, the secure application 416 and/or web platform 418 may facilitate data visualization of collected action data, and other data, and may include a data analysis engine that allows for specification and customization of data visualization. The secure web platform 418, secure application 416, and/or an application running on the personal terminal 410 may use a cross-platform development environment to allow application data to be synchronized across multiple platforms, and devices, simultaneously. Thus, the system 400 may allow enhanced monitoring of patent activities, enhanced reminders for patients of required activities, and enhanced communication between patients and caregivers or medical professionals.

A tag device, wearable device, and personal terminal may communicate to monitor user actions, such as user engagement in activities of daily living, and provide users with alerts. An example system 500 may include a tag device 502, a wearable device 504, and a personal terminal 506. The tag device 502, wearable device 504, and personal terminal 506 may all communicate with each other. In some embodiments, one or more of the tag device 502, the wearable device 504, and the personal terminal 506 may communicate with the cloud 508, such as with a remote server.

Tag devices may be attached to various household items and locations such as doors, showers, bathtubs, toilets, microwaves, freezers, beds, chairs, vehicles, clothing items, and other household items. A tag device 502 may include a controller module 512 for controlling the tag device 502. The controller module 512 of the tag device 502 may, for example, include a processor or embedded controller. The tag device 502 may be programmable to allow users, caregivers, and/or medical professionals to create custom tag applications. In some embodiments, the tags 502 may be programmed using a web platform or mobile application. The tag device may also include a battery 518, such as a lithium ion battery, for powering the tag device 502. The battery 518 may have a lifetime of twelve months. The tag device 502 may also include a sensor module 516 for sensing user input to the tag device 502. For example, the sensor module 516 may include a gyroscope, an accelerometer, a microphone, a button, a switch, or other sensing device. For example, the sensor module 516 may sense movement of the tag device 502, audio received by the tag device 502, or pressing of a button or toggling of a switch of the tag device 502. The tag device 502 may further include a communication module 522 for communicating with a wearable device 504, a personal terminal 506, and/or a server on the cloud 508. The communication module 522 may include a BLE module, a WiFi module, a cellular connectivity module, or other communication technology. In some embodiments, the communication module 522 may include only an RFID tag, and the tag device 502 may not include a controller module 512, a memory 514, a sensor module 516, a battery 518, or an alert module 520, to reduce costs. In such embodiments, the RFID tag of the tag device 502 may be used by the wearable device 504 to detect proximity of the wearable device 504 to the tag device 502. The tag device 502 may also include an alert module 520 for alerting a user. For example, the tag device 502 may receive an alert generation command via the communication module 522 from the wearable device 504, from the personal terminal 506, or from a remote server on the cloud 508. The tag device 502 may communicate with the wearable device 504 via a secure pairing and communications BLE protocol. The alert module 520 may include a speaker, a tactile feedback module, a visual feedback module, such as a display or one or more LEDs, or other alert technology. When the tag device 502 receives an alert generation command, the controller module 512 may cause the alert module 520 to generate an alert. A tag device 502 may also include a memory 514 for storing information. For example, the memory 514 may store proximity information detected when the tag device 502 connects to or detects the wearable device 504, information sensed by the sensor module 516, such as motion or button triggering information, and other information. In some embodiments, the tag device 502 may communicate proximity and/or sensed information with the wearable device 504 and/or the personal terminal 406. In some embodiments, information from the tag device may be communicated to the personal terminal 506 via the wearable device 504.

The wearable device 504 may be worn by a user and may sense motion of the user, proximity of the user to one or more tag devices, and other information related to activity and/or a status of the user. The wearable device 504 may have low power requirements through implementation of a sleep-cycle, where the sensor(s) of the wearable device 504 are configured to enter a sleep mode between sampling intervals. The wearable device 504 may also implement an adaptive movement analysis algorithm to minimize CPU usage during periods of rest, such as sleeping or sitting. The wearable device 504 may include a controller module 526 to control the wearable device, such as a processor or embedded controller. The wearable device 504 may further include a communication module 524. The communication module 524 may include, for example, a Bluetooth module, a BLE module, a WiFi module, a cellular connection module, an RFID reader, or other communication technology. In some embodiments, the communication module 524 of the wearable device 504 may communicate with the communication module 522 of a tag device 502 via a BLE connection, may communicate with a communication module 532 of the personal terminal 506 via a Bluetooth connection, and/or may communicate with a server in the cloud 508 via a cellular connection. A BLE connection between the wearable device 504 and the tag devices 502 may use secure pairing to provide secure data transmission between the tag devices 502 and the wearable device 504. The wearable device may also include a sensor module 530 for sensing movement of the user. The sensor module 530 may include a gyroscope, an accelerometer, a microphone, a button, a switch, or other sensing device. The wearable device 504 may include a battery 534, such as a lithium ion battery for powering the wearable device 504. The battery 534 may be rechargeable. In some embodiments, the battery 534 may have a lifetime of up to twelve months. The wearable device 504 may also include an alert module 532 for alerting a user. The alert module 532 may include a speaker, a tactile feedback module, a visual feedback module, such as a display or one or more LEDs, or other alert technology. The alert module 532 may generate alerts for a user when an alert generation command is received from the personal terminal 506 or from a server on the cloud 508. The wearable device 504 may also include a memory 528 for storing information. The memory 528 may, for example, store information related to proximity detected between the wearable device 504 and a tag device 502, such as by establishment of a BLE connection or detection of an RFID tag in a tag device 502, information transmitted to the wearable device 504 from the tag device 502, such as information sensed by the tag device 502, information received from the personal terminal 506, and/or information sensed by the sensor module 530 of the wearable device 504. For example, the wearable device 504 may store proximity detection information specifying proximity detected by the wearable device 504, such as proximity detected between the wearable device 504 and a tag device 502, in a data log of the memory 528 for transmission to the personal terminal 506. The wearable device 504 may also store input information received from the tag device 502 in the data log of the memory 528 for transmission to the personal terminal 506. The wearable device 504 may transfer activity data, such as proximity data, motion data, and user input data, collected by the wearable device 504 from one or more tag devices 502 or generated by the wearable device 504, to the personal terminal 506. Communication between the wearable device 504 and the personal terminal 506 may be encrypted using an encryption algorithm, such as KP-ABE. In some embodiments, the wearable device 504 may connect to and transfer data to the personal terminal 506 when the wearable device 504 is brought within a certain proximity of the personal terminal 506, such as within five meters.

The personal terminal 506 may be a laptop, tablet, or smart phone device. For example, the personal terminal may be a tablet, such as an Android operating system tablet, configured to operate in a kiosk mode. The personal terminal 506 may be configured to launch an application for the system 500, such as an application for receiving data from and providing data and/or instructions to the tag devices 502 and the wearable device 504, providing reminders to a user, receiving input from a user, facilitating communication between a user and caregivers and/or medical professionals, and transmitting data to a server on the cloud. A user interface of the application may be designed to comply with guidelines for designing user interfaces for older adults, including following recommendations for user interface color, font size, contrast, sharpness, pixel size, and other user interface attributes. In some embodiments, the personal terminal may be configured to monitor proximity between the wearable device 504 and/or the tag devices 502 using a received signal strength indication (RSSI). In some embodiments, communication between the wearable device 504 and the personal terminal 506 may be limited to a predetermined interval, such as every ten seconds, to conserve power. The personal terminal 506 may be configured to automatically pair to tag devices 502 and/or wearable devices 504 that are within range of the personal terminal 506. In some embodiments, the personal terminal 506 may be maintenance free and may prevent user access to operating system settings that might interfere with operation of the personal terminal 506. The personal terminal 506 may have a large touch screen, such as a 14-inch touch screen. The personal terminal 506 may, for example, accept both touch and voice input using touch and voice application programming interfaces (APIs) utilizing WaveNet and neural networks to provide high-fidelity speech synthesis in multiple languages and variants.

The personal terminal 506 may receive activity information, such as proximity detected to a tag device 502, and sensed information, such as user input information, from a wearable device 504 and/or a tag device 502. For example, when the wearable device 504 detects that a tag device 502 is within a predetermined proximity, such as by detecting an RFID tag of the tag device 502 or by forming a BLE connection with the tag device 502 or detecting, using the BLE connection, that the tag device 502 is within a predetermined proximity of the wearable device 504, a communication module 532 of the personal terminal 506 may receive such information, from a communication module 524 of the wearable device 524. The personal terminal 506 may determine, based on received activity information, that a user has performed an action, such as engaging in one or more activities of daily living. For example, if the personal terminal 506 receives proximity information from the wearable device 504 showing proximity between the wearable device 504 and a tag device 502 attached to a refrigerator at 8:00 AM, the personal terminal 506 may determine that the user has eaten breakfast. In some embodiments, received activity information, sensed information, and user input information may be analyzed by an application or operating system 542 executed by a processor of the personal terminal. The personal terminal may also include a user interface 544. The user interface 544 may, for example, include a display, such as a touch screen display, for displaying alerts, such as messages or appointments, for the user, and allowing the user to communicate with caregivers and/or medical professionals. The user interface 544 may also include a microphone for receiving voice data from the user, allowing voice recognition and/or audio communications with medical professionals and/or caregivers. The user interface 544 may include a speaker for providing audio alerts to a user. For example, the personal terminal 506 may emit an audio alert to remind a user to complete a scheduled user action. In some embodiments, the personal terminal 506 may remind a user to complete an action contingent on a detected action. For example, if the personal terminal 506 determines that a user has taken a shower, but has not determined that a user has turned the water off for the shower, the personal terminal may remind the user, via an internal speaker of the user interface 544 or via triggering a reminder notification using the alert module 532 of the wearable device 504, to turn off the water. The personal terminal 506 may also include a battery 546 for powering the personal terminal and a memory 540 for storing data, such as detected user actions and proximity and sensed information received from the wearable device 504 and/or the tag device 502. In some embodiments, the personal terminal 506 may be configured to operate with a constant power supply, such as a supply from a standard power outlet. Data stored within the personal terminal 506, such as within the memory 540 of the personal terminal, may be encrypted using symmetric-key encryption, with each sensor generating a new symmetric key at a predetermined interval, such as daily. A KP-ABE algorithm may be used to encrypt the symmetric key. For example, a cloud-based server in the cloud 508 may transmit a public key to the personal terminal 506, and the personal terminal 506 may encrypt the key and write the result to a log. Because only the server can generate a corresponding private key, only those who are granted access can decrypt the data. Daily rotation of the keys can provide forward and backward protection against compromise of a single key. Furthermore, data transmitted over internet or cellular connections, such as data transmitted from the personal terminal 506 to a server in the cloud 508, may be transmitted using industry-standard encrypted transport security. Because the data is encrypted, compromise of data transmitted will not result in clear text data.

The personal terminal 506 may upload information, such as detected user actions, received activity information, user input information, and other information to a server on the cloud 508. For example, the personal terminal 506 may upload information using the communication module 532 via a wired or wireless internet connection, cellular connection, or other connection. In some embodiments, the personal terminal 506 may notify medical professionals, caregivers, and/or family members, via communication with a server on the cloud 508 when a scheduled action or action that should be completed has not been completed. For example, when a user has not eaten breakfast by a predetermined time, the personal terminal 506 may notify the server on the cloud 508 that the user has not eaten breakfast. The server may push a notification to a secure application or secure web platform accessible by one or more caregivers and/or medical professionals. In some embodiments, the personal terminal 506 may receive instructions to generate reminders for the user from caregivers and/or medical professionals using the secure application or secure web platform.

The cloud 508 may include a scalable cloud architecture including multiple servers for receiving data from and transmitting data to personal terminals 506. For example, the cloud may include a flexible document-oriented NoSQL database software that is scalable, highly available, and partition tolerant. Servers operating on the cloud 508 may include data storage systems with RAID-5 storage redundancy to prevent data loss from a single disk hardware failure. The data storage on the cloud 508 may hierarchically organize and expose resources through uniform interfaces based on the representation state transfer (REST) paradigm. In some embodiments, the cloud 508 may include a dedicated status report server to communicate with status report clients operating on the personal terminals 506 to receive messages regarding system status for multiple managed system terminals 506, wearable devices 504, and/or tag devices 502. The cloud 508 may include auditing functionality, data backup functionality, and disaster recovery functionality in compliance with the Health Insurance Portability and Accountability Act (HIPAA). Data from personal terminals 506 may be received periodically by servers on the cloud 508. A main entry point of the cloud 508 may include a traffic dispatcher that first dispatches received data streams to one or more storage servers. Such an entry point may help to distribute data request traffic to multiple back-end servers providing enhanced scalability and efficiency. The communication between the tag devices 502, the wearable device 504, the personal terminal 506, and the cloud 508 may include live uploading protocols and data-security architectures, such as advanced encryption and secure internet communications protocols. For example, the communications may be designed in compliance with Food and Drug Administration guidelines for medical device software development and quality control, following International Standards Organization standards. Communication between the personal terminal 506 and the cloud 508 may be encrypted using an encryption algorithm, such as KP-ABE. Furthermore, data access via the cloud using a secure application or web platform may also be encrypted. Thus, tag devices and wearable devices may be used in conjunction with a personal terminal and server on the cloud to track user actions and to provide reminders to a user regarding required actions.

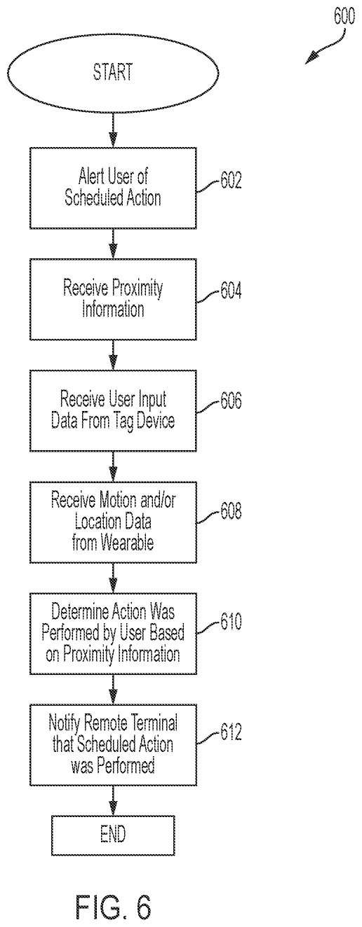

A system including a wearable device, one or more tag devices, and a personal terminal may notify users of scheduled actions and may monitor user activity and actions. An example method 600 for user notification and monitoring, shown in FIG. 6, may begin, at step 602, with alerting a user of a scheduled action. For example, a personal terminal may emit an audio alert or display a visual notification of a scheduled action. For example, a personal terminal may be configured to emit an audio reminder for a user to eat breakfast at 8:00 AM every day. In some embodiments, a personal terminal may transmit an alert generation instruction to a wearable device or a tag device, to cause the wearable device or tag device to display, emit, or otherwise generate an alert. For example, a wearable device may generate tactile feedback, such as vibration, to remind a user. In some embodiments, the personal terminal, wearable device, or tag device may receive an instruction to generate an alert from a control terminal, such as from a server in the cloud. For example, a medical professional or caregiver may generate an alert by transmitting a message to a cloud-based server, which may then be forwarded to a personal terminal, wearable device, and/or tag device of the user. In some embodiments, the wearable device may alert a user of an action that is required based on a detected action. For example, if the personal terminal detects that the user took a shower but did not turn the water off or used the toilet but did not flush the toilet, the personal terminal may alert the user that the water must be turned off or the toilet must be flushed. If the personal terminal detects that the user cooked breakfast, but did not turn the stove off, via a lack of motion information received from a tag device attached to a controller of the stove, the personal terminal may alert the user that the stove needs to be turned off. In some embodiments, the personal terminal may cause the alert to be generated until a determination is made that a scheduled or required action is performed by the user.

At step 604, proximity information may be received. For example, a personal terminal may receive proximity information from a wearable device worn by the user and/or from one or more tag devices. For example, tag devices may be attached to various household objects, such as doors, toilets, showers, bathtubs, faucets, refrigerators, dining tables, chairs, beds, water bottles, phones ovens, microwaves, prescription medications, and other household objects. Proximity may be detected using a wireless connection between the wearable device and one or more tag devices. For example, the wearable device and tag devices may include embedded Bluetooth or Bluetooth low energy (BLE) connectivity. Proximity may be detected when a connection is formed between the wearable device and one or more tag devices, or when a connection, such as a BLE connection, between the wearable device and a tag device determines that the wearable device is within a predetermined distance of the tag device. In some embodiments, tag devices may include RFID tags and the wearable device may include an RFID tag reader. Proximity between the wearable device and a tag device may be detected by the wearable device when the wearable device detects an RFID tag of the tag device. A wearable device worn by a user, such as around a neck of a user or on a wrist or ankle of a user, may transmit proximity information regarding proximity of the wearable device to one or more tag devices to a personal terminal. In some embodiments, one or more tag devices may transmit proximity information to the wearable device to the personal terminal in place of, or in addition to, transmission of proximity information by the wearable device. In some embodiments, proximity information may be transmitted to and received by a remote server. In some embodiments, proximity information may include video and/or image input data from one or more cameras. For example, video and/or image data of a refrigerator may be analyzed to detect motion of the user opening and/or closing the refrigerator.

In some embodiments, at step 606, user input data may be received from a tag device. For example, tag devices may include motion sensors, such as an accelerometer or a tri-axial gyroscope, microphones, buttons, switches, and other sensors to sense user input. For example, a tag device may include motion sensors to sense movement of the tag device for determining whether a user has performed an action. For example, a tag device with motion sensing capabilities may be attached to a handle of a toilet to determine if the toilet has been flushed, to a door of a refrigerator to determine if a refrigerator door has been opened, to a prescription medication container to determine when the user has taken the prescription medication, or to a faucet handle of a bathtub or shower to determine when water is turned on and/or off. In some embodiments, a tag device may include one or more buttons or switches to be pushed or toggled by a user. For example, a tag device attached to a prescription medication container may include a button to be pushed by the user every time the user takes the medication. User input data gathered from one or more sensors of the tag devices may be transmitted directly to a personal terminal device, via a Bluetooth or WiFi connection, or to a wearable device, through a Bluetooth or Bluetooth low energy connection, and then from the wearable device to the personal terminal. In some embodiments, user input data may be transmitted from tag devices to a remote server via a cellular or WiFi connection. Sensed data, such as user input data, may be received by the wearable device from one or more tag devices and may be transmitted to the terminal device from the wearable device. For example, the wearable device may collect sensed data from multiple tag devices that a user has interacted with and may transmit the sensed data to the terminal for analysis to determine user actions. Such sensed data may, for example, be included in a data log along with proximity sensing information transmitted from the wearable device to the terminal device and may be used to determine whether a user has engaged in one or more actions. Collection of sensed data by a wearable device may reduce a cost of the system by reducing the number and cost of communications components included in a tag device. For example, longer-range wireless communications components such as cellular connection modules and/or Wi-Fi modules may not need to be included in a tag device as the tag device may communicate with the wearable device via a NFC or Bluetooth connection. Furthermore, NFC and/or Bluetooth communications modules may use less power than Wi-Fi or cellular communications modules, enhancing a battery life of tag devices when sensed data is collected from tag devices by the wearable. The collected sensed data may enhance an accuracy of determination that one or more actions were taken by a user when combined with proximity information, and used to verify that the received proximity information correctly indicates completion of an action. For example, sensed data from a tag attached to a shower handle may enable confirmation that a user turned on the shower, in addition to being in proximity to the shower. Thus, sensed data including user input data may also be used in determining whether one or more actions have been completed.

In some embodiments, at step 608, motion and/or location data may be received from the wearable device. Motion data may for example, include motion data sensed by motion sensors of the wearable device, such as a tri-axial gyroscope and/or accelerometer. Location data may include global positioning system (GPS) or other location data received from the wearable device. Motion and/or location data may be received by a personal terminal via a wireless connection between the wearable device and the personal terminal, such as a Bluetooth or WiFi connection. Alternatively or additionally, motion and/or location data may be received by a remote server via a wireless connection, such as an WiFi or cellular connection. In some embodiments, motion and/or location data may be received from another device, such as a smart phone of the user.

At step 610, a determination may be made, based on the received proximity information, that an action has been performed by the user. In some embodiments, the action may be an action for which an alert was generated at step 602. For example, when proximity information indicating that the wearable device was in proximity with a tag device attached to a shower is received, a determination may be made that the user of the wearable device has taken a shower. The determination may be made by a personal terminal or by a remote server in the cloud. In some embodiments, the determination may be made based on received user input information in addition to or in place of proximity information. For example, a determination that a medication was taken may be made based on received proximity information indicating proximity between the wearable device and the tag device attached to the medication and user input information indicating the user pressed a button on the tag device. Thus, the personal terminal, or a remote server, may determine that a user performed a user action based on received proximity information. In some cases, a determination may be made that an action has been completed before a scheduled time for the task and/or before a scheduled reminder for the action was displayed, emitted, or otherwise delivered to the user. In such cases, a scheduled reminder may be cancelled based on the determination that the action has already been performed.

In some embodiments, a determination may be made, based on proximity information or other information, that a user has performed an action outside the home. For example, a determination may be made that a user has attended a medical appointment or gone grocery shopping. Such a determination may be made based on location information received from the wearable device, from proximity information regarding proximity between the wearable device and one or more tag devices at medical offices or grocery stores, or from GPS data, such as GPS data received from a smart phone of the user.

In some embodiments, the determination, at step 610, that an action has been performed by the user may include determining a life space size of the user. Determination of an action may, for example, include a determination that a user has entered a room of the user's residence. For example, a determination may be made, based on received proximity information, that a user has entered one or more rooms of a user's residence. Alternatively or additionally, a determination may be made that a user has visited one or more remote locations outside of a user's residence. In some embodiments, the determination, at step 610, that an action has been performed may include determinations of multiple actions, such as entering of multiple rooms of a user's residence by the user. A life space of the user may be determined based on the number of rooms visited by the user in a predetermined time period, such as a day. In some embodiments, a life space of the user may, for example be an estimated area during which the user operates daily, and may be calculated based on a number of rooms visited by the user in a one day period and/or based on a greatest distance traveled by the user in a day. In some embodiments, a cognitive state of the user, such as a level of cognitive impairment, may be determined based on a determination of a life space of the user. A determination of a cognitive state of the user may be determined by a remote server or by the personal terminal. For example, a determination may be made that a user is in good cognitive health if a life space of the user is large, remains constant, and/or expands. A determination that a user is in poor cognitive health, or is experiencing a high or increasing level of cognitive impairment, may be made if a life space of the user is small or shrinking.

In some embodiments, the determination, at step 610, that an action has been performed by the user may include a determination that one or more activities of daily living have been completed by the user. For example, the determination may be made that the user has bathed and/or that the user has prepared and consumed a meal. In some embodiments, a determination that an activity of daily living has been completed may include determination that multiple actions have been completed by a user based on received proximity information. For example, a determination that a user has eaten a meal may include determinations that a user has accessed a pantry, based on received proximity information between the wearable device and a tag on a pantry door, and that the user has used a microwave, stove, or oven, based on received proximity information between the wearable device and a tag on the microwave, stove, or oven. In some embodiments, a determination of a cognitive state of the user may be made based on the determination that one or more activities of daily living have been performed. For example, if a user consistently performs activities of daily living with minimal or no reminders, a determination may be made that the user is in good cognitive health. However, is a user fails to perform activities of daily living, or requires repeated reminders to perform activities of daily living, a determination may be made that the user is in poor or declining cognitive health. In some embodiments, performance of activities of daily living may be analyzed over periods of weeks or months to monitor user cognitive health over time and detect improving or declining cognitive health.

In some embodiments, the personal terminal may, at step 612, notify a remote terminal, such as a server on the cloud, that a scheduled action was performed. In some embodiments, the personal terminal may notify the remote terminal of actions that are performed even if the actions are not scheduled actions. For example, when a determination is made by the personal terminal that a user has eaten breakfast, a notification may be transmitted to a remote server notifying the server that the user has eaten breakfast. The remote server may then push the notification to an application or secure web platform accessible by one or more caregivers or medical professionals to notify the caregivers or medical professionals that the action was performed. Aggregated action information may provide medical professionals with data that may help track cognitive decline, predict future deterioration in action performance, and predict frailty and fails. Thus, a system for user monitoring and reminders may enhance the quality of care provided by caregivers and medical professionals, by providing enhanced user activity information, and may also provide greater peace of mind to family members of users.

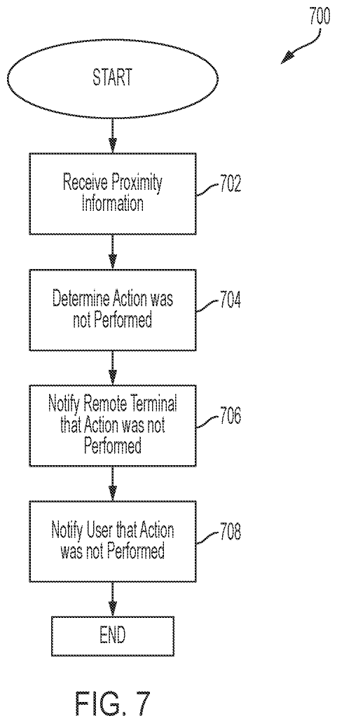

In some embodiments, users, such as patients with chronic aging diseases, such as dementia and Alzheimer's, may forget to perform required or scheduled actions. In such cases, reminders for the user of required actions and/or notifications to caregivers and/or medical professionals that such actions have not been performed may enhance quality of care for the user. An example method 700 of determining that an action was not performed and alerting a user, a caregiver, and/or a medical professional of the failure to perform a task is shown in FIG. 7. At step 702, proximity information may be received. As discussed with respect to step 604, proximity information from a wearable device and/or a tag device may be received by a personal terminal or a remote server on the cloud. For example, proximity information may be received indicating that a wearable of a user was in proximity with a tag device attached to a shower. In some embodiments, a failure to perform a scheduled action, or an action requested by a caregiver or medical professional, may be determined by a lack of receipt of proximity information.

At step 704, a determination may be made that an action was not performed. The determination may be made based on received proximity information, received user input information, and/or a lack of received proximity information and/or user input information. For example, if a user was scheduled to take a prescription medication, and proximity information showing proximity between a wearable and a tag device attached to a container of the prescription medication and/or user input information from the tag device is not received by a predetermined time, a determination may be made that the user failed to take the medication. As another example, if proximity information is received indicating a user has taken a shower, but input information, such as motion information from a tag device attached to a faucet handle of the shower, indicating that the water in the shower has been turned off is not received, a determination may be made that the user has failed to turn the shower water off. The determination may be made by a personal terminal device, such as a laptop or tablet, or by a remote cloud server.

At step 706, a remote terminal may be notified that a required or scheduled action was not performed. For example, a personal terminal may determine that a user has not performed a scheduled action by a predetermined time, or that a user has not performed a required action, such as an action required following a detected action, within a predetermined time. For example, if the personal terminal determines that a user has failed to turn the water off after detecting that the user has taken a shower, the personal terminal may transmit a notification that the user has not turned the water off to a remote server. As another example, if the personal terminal determines that the user has failed to eat breakfast by a predetermined time, due to a lack of receipt of proximity information between the wearable device and a tag device attached to a refrigerator, the personal terminal may notify a remote terminal that the user has not eaten breakfast. In some embodiments notifying the remote terminal may include transmitting a notification to a remote server on the cloud. Alternatively or additionally, the notification may be transmitted to an application or secure web-based platform on a smart phone, tablet, desktop or other device operated by a caregiver, medical professional, or family member that the user has not completed a scheduled or required action. In some embodiments, a remote server may push notifications to smart devices operated by caregivers, medical professionals, and/or family members. Such notifications may enhance treatment by allowing caregivers and/or medical professionals to take action if a critical action is not performed. Notifications of activities performed and failure to perform activities may also enhance care by providing better patient activity datasets through logging of activities performed and missed. In some embodiments, a notification that a required or scheduled action was not performed may include a notification that one or more activities of daily living were not performed. In some embodiments, a determination may be made that a user is in poor or declining cognitive health may be made based on failure, or repeated failure, to perform one or more activities of daily living. Notifications of determined poor or declining cognitive health may be transmitted to caregivers and/or medical professionals to allow for improved care management.

At step 708, a user may be notified that a required action was not performed. For example, the personal terminal may emit an audio alert, display a visual alert, and/or generate a tactile alert to inform the user that a required or scheduled action has not been performed. The personal terminal or remote server may also cause a wearable device or one or more tag devices to emit, display, or generate an alert to notify the user that a scheduled or required action has not been performed. For example, if an action of eating breakfast was scheduled to be performed by 8:30 AM and the personal terminal or remote terminal determines that the user has not eaten breakfast by 8:30 AM, the personal terminal or remote terminal may cause a message to appear on a display of the personal terminal and/or an audio message to be played through a smart speaker connected to the personal terminal reminding the user to eat breakfast. As another example, if a user has forgotten to turn the water off after taking a shower, the personal terminal or remote terminal may cause the wearable device to vibrate or emit an audio alert, such as a tone or message, instructing the user to turn the shower water off. In some embodiments, the notification may continue until a determination has been made that the user has completed the scheduled or required action. Thus, a system including a wearable device, a personal terminal, and one or more tag devices may also help to remind a user to perform forgotten scheduled and/or required tasks.

In some embodiments, the sequences of activities performed after a reminder, either voice, text, or other reminder, is used to schedule future reminders. For example, after a reminder for taking a medication, interaction between the user and the tagged box of medication is observed and used to determine whether a successful response to the reminder is obtained. If no successful response was detected within a pre-defined time interval, another reminder is sent. After several unsuccessful responses, a reminder may be sent to a caregiver and/or a care provider. In some embodiments, the number of successful responses or changes in number of successful responses over time is used to assess cognitive function of the user.

The schematic flow chart diagrams of FIGS. 6-7 are generally set forth as a logical flow chart diagram. As such, the depicted order and labeled steps are indicative of aspects of the disclosed method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagram, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The operations described above as performed by a controller may be performed by any circuit configured to perform the described operations. Such a circuit may be an integrated circuit (IC) constructed on a semiconductor substrate and include logic circuitry, such as transistors configured as logic gates, and memory circuitry, such as transistors and capacitors configured as dynamic random access memory (DRAM), electronically programmable read-only memory (EPROM), or other memory devices. The logic circuitry may be configured through hard-wire connections or through programming by instructions contained in firmware. Further, the logic circuitry may be configured as a general purpose processor capable of executing instructions contained in software. If implemented in firmware and/or software, functions described above may be stored as one or more instructions or code on a computer-readable medium. Examples include non-transitory computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise random access memory (RAM), read-only memory (ROM), electrically-erasable programmable read-only memory (EEPROM), compact disc read-only memory (CD-ROM) or other optical disk storage, magnetic disk storage or other magnetic storage devices, or any other medium that can be used to store desired program code in the form of instructions or data structures and that can be accessed by a computer. Disk and disc includes compact discs (CD), laser discs, optical discs, digital versatile discs (DVD), floppy disks and Blu-ray discs. Generally, disks reproduce data magnetically, and discs reproduce data optically. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims.

Although the present disclosure and certain representative advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A system, comprising:
a wearable device; and
a personal terminal,
wherein the personal terminal comprises a processor configured to perform steps comprising:
receiving, from the wearable device, proximity detection information, the proximity detection information identifying proximity between the wearable device and a tag device;
receiving, from the wearable device, sensor data received by the wearable device from the tag device; and
determining, based on the proximity detection information and the sensor data, that a first action was performed by a user.

2. The system of claim 1, wherein the sensor data comprises motion data indicative of motion of the tag device.

3. The system of claim 2, wherein:
the motion data comprises motion data indicating adjustment of a water faucet; and
determining, based on the proximity detection information and the sensor data, that a first action was performed by a user comprises determining that the user has taken a shower based on the proximity detection information and the sensed motion data indicating adjustment of a water faucet.

4. The system of claim 1, wherein the processor is further configured to perform steps comprising:
alerting the user of a scheduled action,
wherein the step of determining comprises determining that the scheduled action was performed by the user.

5. The system of claim 1, wherein the processor is further configured to perform steps comprising:
determining, based on the proximity detection information, that a second action was performed by the user;
determining, by the personal terminal based on the determination that the first action was performed and based on the determination that the second action was performed, that an activity of daily living was performed by the user.

6. A method, comprising:
receiving, by a personal terminal from a wearable device, proximity detection information, the proximity detection information identifying proximity between the wearable device and a tag device;
receiving, by the personal terminal from the wearable device, sensor data received by the wearable device from the tag device; and
determining, by the personal terminal based on the proximity detection information and the sensor data, that a first action was performed by a user.

7. The method of claim 6, wherein the sensor data comprises motion data indicative of motion of the tag device.

8. The method of claim 7, wherein the sensed motion data comprises sensed motion data indicating adjustment of a water faucet, and wherein determining, based on the proximity detection information and the sensor data, that a first action was performed by a user comprises determining that the user has taken a shower based on the proximity detection information and the sensed motion data indicating adjustment of a water faucet.

9. The method of claim 6, further comprising:
receiving at least one of motion data or location data sensed by the wearable device,
wherein the determination that the first action was performed by the user is further based on the received motion data or location data.

10. The method of claim 9, further comprising:
alerting, by the personal terminal, the user of a scheduled action,
wherein the step of determining comprises determining that the scheduled action was performed by the user.

11. The method of claim 6, further comprising determining, by the personal terminal, a life space size of the user based on the proximity detection information.

12. The method of claim 11, further comprising determining a cognitive state of the user based on the determination of the life space size of the user.

13. The method of claim 6, further comprising:
determining, by the personal terminal based on the received proximity detection information, that a second action was performed by the user;
determining, by the personal terminal based on the determination that the first action was performed and based on the determination that the second action was performed, that an activity of daily living was performed by the user.

14. The method of claim 13, further comprising determining a cognitive state of the user based on the determination that the activity of daily living was performed.

15. An apparatus, comprising:
a processor; and
a memory,
wherein the processor is configured to perform steps comprising:
receiving, from a wearable device, proximity detection information, the proximity detection information identifying proximity between the wearable device and a tag device;
receiving, from the wearable device, sensor data received by the wearable device from the tag device; and
determining, based on the proximity detection information and the sensor data, that a first action was performed by a user.

16. The apparatus of claim 15, wherein the sensor data comprises motion data indicative of motion of the tag device.

17. The apparatus of claim 16, wherein the sensed motion data comprises sensed motion data indicating adjustment of a water faucet, and wherein determining, based on the proximity detection information and the sensor data, that a first action was performed by a user comprises determining that the user has taken a shower based on the proximity detection information and the sensed motion data indicating adjustment of a water faucet.

18. The apparatus of claim 15, further comprising
receiving at least one of motion data or location data sensed by the wearable device,
wherein the determination that the action was performed by the user is further based on the received motion data or location data.

19. The apparatus of claim 15, wherein the processor is further configured to perform steps comprising:
alerting, by the personal terminal, the user of a scheduled action,
wherein the step of determining comprises determining that the scheduled action was performed by the user; and
notifying, by the personal terminal, a remote terminal that the action was performed by the user.

20. The apparatus of claim 15, wherein the step of determining that an action was performed by a user comprises determining that the user entered one or more predetermined rooms, and wherein the processor is further configured to perform steps comprising determining a life space size for the user based, at least in part, on the determination that the user entered the one or more predetermined rooms.
